(19) United States
(12) Patent Application Publication
(10) Pub. No.: US 2006/0254916 A1
(43) Pub. Date: Nov. 16, 2006

(54) Methods of forming microfluidic devices and uses thereof

(76) Inventors: Juan J. Hernandez, San Diego, CA (US); Eric Lee Burch, San Diego, CA (US); Gary Larson, San Diego, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/130,698

(22) Filed: May 16, 2005

(51) Int. Cl. C07K 1/26

(52) U.S. Cl. 204/453

(57) ABSTRACT

One microfluidic device of the present invention includes a substrate having a surface and a depression formed in the surface of the substrate. The microfluidic device further includes a detectable marker in the depression and a coating covering the surface. Methods of producing and using the microfluidic device are further described.

### TECHNICAL FIELD

The present invention relates generally to microfluidic devices, and more particularly to methods for containing reagents in microfluidic devices for chemical or optical analysis, and methods for producing the microfluidic devices.

### BACKGROUND OF THE INVENTION

In microfluidic chips, the sample being analyzed is mixed with a variety of chemicals in order to modify the sample or determine the sample's chemical properties. Known analytical methods employing microfluidic chips use buffers and chemical reservoirs that are located external to the chip, use coupling agents, or use packaged dry chemicals. Another known method utilizes membranes that selectively transport analytes, such as in the case of electrodes.

However, each of the methods currently in use suffers from drawbacks. For instance, chips having external ports for adding chemicals require expensive ancillary support and control equipment such as pressure and flow control equipment. Further, the added chemicals may generate a significant amount of waste in addition to the chemicals actually needed for the sample analysis.

If coupling agents are used, they are often difficult to apply to specific areas of the microfluidic chip since the coupling agents usually require spraying or immersing of the entire sample to get the coupling agent in the specific area. The coupling agents may also be very difficult to control since they are often only one monolayer thick and push the limits of ellipsometry. Further, the ability of the coupling agents to bond to substrates depends on the uniformity of the layer of the coupling agent and the compound used for chemical termination, such as —COOH or —NH₃. Thus, the placement of multiple reagents in a chip is often challenging and expensive.

When packed dry chemicals are used, the dry chemicals are often carried with the eluent as the eluent flows over the dry chemicals. This results in a potential mixing of the dry chemicals with other chemicals and may lead to difficult optical control of the sample. With regard to the use of osmotic membranes, they are often expensive and present challenges for integration into microfluidic chips.

### SUMMARY OF THE INVENTION

In one embodiment, a microfluidic device is disclosed. The microfluidic device comprises a substrate having a surface and a depression formed in the surface of the substrate. The microfluidic device further includes a coating and at least one detectable marker anchored in the coating.

In another embodiment, a method for producing a microfluidic device comprises forming a channel in a surface of a substrate and placing a mixture comprising polyvinyl alcohol on at least a portion of the surface of the substrate. A detectable marker is anchored in the mixture.

In an additional embodiment, a method for analyzing a sample includes forming a depression in a surface of a substrate and placing a coating on at least a portion of the surface of the substrate. A detectable marker is anchored in the coating, a sample is placed in the depression, and the sample is analyzed with the detectable marker.

### BRIEF DESCRIPTION OF THE DRAWINGS

While the specification concludes with claims particularly pointing out and distinctly claiming that which is regarded as the present invention, the advantages of the invention may be more readily ascertained from the following description of the invention when read in conjunction with the accompanying drawings in which:

FIG. 1 is a perspective view of one embodiment of a microfluidic device of the instant invention;

FIG. 2 is a perspective view of one embodiment one of the layers of the microfluidic device of FIG. 1;

FIG. 3 is a cross-sectional view of the microfluidic device of FIG. 1;

FIG. 4 is a flow-chart of one embodiment of a method of forming a microfluidic device of the instant invention; and

FIG. 5 is a schematic diagram of one embodiment of a system for analyzing a sample using a microfluidic device of the instant invention.

### DETAILED DESCRIPTION OF THE INVENTION

In each of its various embodiments, the present invention is directed to microfluidic devices, methods for producing the microfluidic devices, and methods for analyzing a sample using the microfluidic device. The microfluidic devices may be used by analytical systems employing optical analysis, such as ultra-violet light, infrared light, fluorescent light, absorbance, transmittance, reflectance, color content, or other optical analysis tools. A variety of qualitative and/or quantitative analyses of the sample may be performed. Analytical systems in which the microfluidic devices may be used include, without limitation, a spectrophotometer, a mass spectrometer, or other optical analytical system. Assays that may be performed with the microfluidic device described herein include, without limitation, chromatographic capture, immunoassays, competitive assays, DNA or RNA binding assays, fluorescence in situ hybridization (FISH), protein and nucleic acid profiling assays, sandwich assays, drug discoveries, drug interaction with other compounds, or other known chemical assays.

The method for producing the microfluidic devices described herein is a simple and cost effective process for containing and retaining multiple reagents in a microfluidic device. The method for producing the microfluidic device generates less waste, retains the reagents and fluid (i.e., sample or eluent) under flow in place in the microfluidic devices, and provides a microfluidic device that produces a slow and stable change, such that suitable optical properties ideal for rapid optical measurements are achieved. Use of the microfluidic device enables the quantity of reagent per unit volume of sample to be controlled, thus enabling the analysis of more controlled titration studies. Further, the use of the microfluidic device described herein does not chemically interfere with the sample.

In one embodiment, the microfluidic device includes a substrate, a channel formed in a surface of the substrate, an analytical chamber formed in the surface of the substrate and a material that seals the analytical chamber. In another embodiment, the material that seals the analytic chamber may be used as an adhesive to adhere another substrate, plate, a layer of material or other structure to the substrate. Further, the material that seals the analytic chamber may be used to adhere multiple substrates, plates, layers of materials, other structures of combinations of any thereof together with the substrate.

In other embodiments, the microfluidic device may further include a sensor for analyzing a sample, metallic layers or conductive traces operatively connected to the sensor, a reagent for the analysis of the sample, and any combinations thereof. The sensor may also be operatively connected with circuitry that allows various electric tests to be performed.

In one embodiment, the substrate is a material such as fused silica, glass, ceramic, metal, plastic, silicon, paper, a silica material, soda lime, various plastics or any combination thereof, wherein the substrate may optionally be rigid. In other embodiments, the substrate may be a material used in the manufacture of transparent or opaque support material. Non-limiting examples include, but are not limited to, clear films, such a cellulose esters, including cellulose triacetate, cellulose acetate, cellulose proprionate, or cellulose acetate butyrate, polyesters, including poly(ethylene terephthalate), polyimides, polycarbonates, polyamides, polyolefins, poly(vinyl acetals), polyethers, polyvinyl chloride, and polysulfonamides. Polyester film supports, and especially poly(ethylene terephthalate), such as manufactured by du Pont de Nemours under the trade designation of MELINEX, may be used because of their excellent dimensional stability characteristics. Opaque materials include, for example, baryta paper, polyethylene-coated papers, and voided polyester. Resin-coated paper or voided polyester may also be used. Other materials, such as transparent films for overhead projectors, may also be used for the support material. Examples of such transparent films include, but are not limited to, polyesters, diacetates, triacetates, polystyrenes, polyethylenes, polycarbonates, polymethacrylates, cellophane, celluloid, polyvinyl chlorides, polyvinylidene chlorides, polysulfones, and polyimides.

The embodiments disclosed herein are efficacious when used with high-gloss film and transparency substrates, as these materials are known to be difficult to coat and adhere to, inasmuch as their surface is very smooth, which results in a small interface area between the coating and the substrate (or subbing layer) and reduced mechanical interlocking adhesion.

Thus, the use of certain support materials, such as polyesters, may be combined with the use of a subbing layer which improves the bonding of the channel coating, described below, to the support. Useful subbing compositions for this purpose are well known in the art and include, for example, terpolymers of vinylidene chloride, acrylonitrile, and acrylic acid or of vinylidene chloride, methyl acrylate, itaconic acid, and natural polymers such as gelatin.

In yet another embodiment, the coating is formed on the substrate (or subbing layer, as the case may be) and may include one or more binders and one or more pigments (fillers). The fillers may be used to increase the speed of liquid adsorption, or can be used to adjust mechanical properties, for example.

In one embodiment, the channel is formed in the substrate by etching, such as reactive ion etching, isotropic etching, or wet or dry chemical etching. In other embodiments, the channel may be formed by mechanical abrasion, laser ablation, grinding, microfabrication, micromachining, water jet, mechanical cutting, abrasion or drilling, photolithography techniques, photo patterning, wet etching, plasma dry etching, or other process for removing material from the substrate.

In another embodiment, the channel or the analytic chamber is configured to have a sensor placed therein for analysis of a sample. The sample may be a chemical, biochemical or biologic fluid, and the sensor, the analytic chamber or the channel may have a plurality of chemical, biochemical, or biological moieties attached thereto, including, but not limited to, a nucleic acid such as DNA or RNA, a protein, a reagent, or any combination thereof. The moiety may be attached to a surface of the channel, the analytic chamber or the sensor through use of a chemical, biochemical or biological array. Further, the amount of the moiety attached to the surface of the channel, the analytic chamber or sensor will vary depending on the type of assay to be performed and/or a quantity of the sample to be tested, and may be determined using routine experimentation known by those of ordinary skill in the art. In another embodiment, a preloaded quantity of the moiety may be used.

The channel or the analytic chamber may have a depth of from a few microns up to several millimeters, depending on a desired need of the microfluidic device. It will be apparent by those of ordinary skill in the art, that the analytic chambers in the microfluidic device may be formed to be proportionally “deeper” than the channels, that is, the analytic chambers may extend deeper in a surface than the channels in order to hold more sample for the analysis of the sample.

In another embodiment, a dye, label, tag, reagent, fluorophore, any other detectable tag, or any combination thereof may be attached to the surface of the channel, the analytic chamber, the sensor, the chemical, biochemical or biological moiety, or any combination thereof for use in analyzing the sample. The quantity of the dye, label, tag or reagent used should be appropriate for the specific application or assay for which the microfluidic device is designed and may be determined using routine experimentation known by those of ordinary skill in the art. Further, when the microfluidic device includes more than one analytic chamber, channel, or combinations thereof, different detectable markers or moieties may be placed in the different analytic chambers or channels such that more than one test may be performed on a sample using a single microfluidic device.

In a particular embodiment, the microfluidic device is configured to substantially seal or encapsulate the channel, the analytic chamber, the sensor, or a combination thereof. The material used to seal the microfluidic device may be flexible and compatible with any fluids or compounds to which the microfluidic device may come in contact. Although the microfluidic device is substantially sealed, the microfluidic device may also have an opening such that a sample may be loaded and subsequently contact the channel and/or sensor. The opening may comprise a port or other entrance that is conveniently located for introducing fluid into the chamber.

In one embodiment, the microfluidic device is produced by a process that includes obtaining a substrate having a channel and, optionally, an analytical chamber formed in a surface thereof, and placing a reagent or detectable marker in the channel and/or the analytical chamber. The channel and/or the analytical chamber is substantially coated with a coating over a surface of the microfluidic device. In one embodiment, the coating comprises a mixture including polyvinyl alcohol. In other embodiments, the coating is water-soluble, water-dispersible, water swellable, solvent soluble, or solvent dispersable. The coating may be a film-forming polymer, natural or synthetic. The amount of coating may range from about 5 to 100 wt %.

Examples of water-soluble polymers useful as coatings include, for example: natural polymers or modified products thereof such as albumin; gelatin; casein; starch; gum arabic; sodium or potassium alginate; hydroxyethylcellulose; carboxymethylcellulose; α-, β-, or γ-cyclodextrin; and the like. In the case where one of the water-soluble polymers is gelatin, all known types of gelatin may be used, such as, for example, acid pigskin or limed bone gelatin, acid- or base-hydrolyzed gelatin, as well as derivatized gelatins such as phthalaoylated, acetylated, or carbamoylated gelatin or gelatin derivatized with the anhydride of trimellytic acid. One natural binder is gelatin.

Synthetic polymers are also used and include, but are not limited to, completely or partially saponified products of copolymers of vinyl acetate and other monomers; homopolymers of or copolymers with monomers of unsaturated carboxylic acids such as (meth)acrylic acid, maleic acid, crotonic acid, and the like; and homopolymers of or copolymers with vinyl monomers of sulfonated vinyl monomers such as vinylsulfonic acid, styrene sulfonic acid, and the like. Additional synthetic polymers include: homopolymers of or copolymers with vinyl monomers of (meth)acrylamide; homopolymers or copolymers of other monomers with ethylene oxide; polyurethanes; polyacrylamides; water-soluble nylon-type polymers; polyvinyl pyrrolidone; polyesters; polyvinyl lactams; acrylamide polymers; substituted polyvinyl alcohol; polyvinyl acetals; polymers of alkyl and sulfoalkyl acrylates and methacrylates; hydrolyzed polyvinyl acetates; polyamides; polyvinyl pyridines; polyacrylic acid; copolymers with maleic anhydride; polyalkylene oxides; methacrylamide copolymers; and maleic acid copolymers. All of these polymers can also be used as mixtures.

The coating may be crosslinked to increase film durability. Crosslinkers include, but are not limited to, boric acid and its derivatives, formaldehyde and its derivatives, etc.

In various embodiments, the coating may also contain, in addition to the binder and pigment, a crosslinking agent for the binder, as well as fillers, natural or synthetic polymers or other compounds well known to someone skilled in this art to improve the physical properties of the coating, such as for example UV absorbers, optical brighteners, light stabilizers, antioxidants, humefactants, surfactants, spacing agents, plasticizers, and the like.

The pigment in the coating may comprise any number of pigment materials well known in this art to make the coating opaque or more adsorptive. Examples of suitable inorganic pigments include, but are not limited to, precipitated calcium carbonate, ground calcium carbonate, kaolin, talc, calcium sulfate, barium sulfate, titanium dioxide, zinc oxide, zinc sulfide, zinc carbonate, satin white, aluminum silicate, diatomaceous earth, calcium silicate, magnesium silicate, synthetic amorphous silica, colloidal silica, fumed silica, precipitated silica, colloidal alumina, pseudo-boehmite, aluminum hydroxide, alumina, fumed alumina, modified aluminas, lithopone, zeolite, hydrated halloysite, magnesium carbonate, and magnesium hydroxide. Examples of suitable organic pigments include, but are not limited to, styrene plastics pigment, acrylic plastics pigment, polyethylene, microcapsules, urea resin, and melamine resin. The pigment is generally present in amounts ranging from 0-90%.

In a further embodiment, the reagent may be added to the mixture used to form the coating. In one embodiment, the mixture to form the coating includes water at varying concentrations depending on a desired thickness of a layer of the coating. The mixture used to form the coating may be placed on the substrate with a micropipette or dispensed with an ink-jet pen, depending on the desired precision and pattern of the mixture used to form the coating on the substrate. In other embodiments, the mixture used to form the coating may be placed on the substrate by spin coating with a screen, by extrusion coating, by dip coating and/or drawing the coating by capillary action, by applying the mixture as a blanket over the substrate and drawing the mixture over the substrate with a bar to a desired thickness, and, optionally, premasking or subsequently patterning the mixture on the substrate. Further, patterning the mixture may be performed in a manner such that a plurality of microfluidic devices are patterned on a single substrate and subsequently singulated into the plurality of microfluidic devices.

The mixture of water and polyvinyl alcohol may be subjected to a heating process, such as baking, to substantially remove the water and form the layer of polyvinyl alcohol. The layer of polyvinyl alcohol may be from about 10 to 50 microns thick, but may be formed to any desired thickness depending on the use of the microfluidic device. For analysis, a sample is placed in an opening in the microfluidic device, wherein the opening is operatively connected to the channel.

In a further embodiment, a sensor and, optionally, a metallic layer, and a conductive trace or circuitry are also integrated in the microfluidic device and operatively connected to the sensor such that the microfluidic device may operatively be connected to a microprocessor, such as a personal digital assistant or a computer for rapid collection, storage or analysis of data that is acquired.

In an additional embodiment, the substrate may be configured with a plurality of channels, a plurality sensors, a plurality of analytic chambers, and a plurality of reagents. The plurality of samples may be analyzed on the same microfluidic device. Each of the plurality of the sensors and the each of the plurality of the reagents may be designed for a single analytic test or for different analytic tests.

In other embodiments, the microfluidic device described herein may be combined with other components used in the microfluidic industry, such as mixers, diffusion chambers, reservoirs, integrated electrodes, pumps, valves, or other microfluidic industry devices, in order to form a lab-on-chip.

### EXAMPLES

The following examples describe various embodiments of methods for producing microfluidic devices, the microfluidic devices, and uses of the microfluidic devices so produced in accordance with the present invention. The examples are merely illustrative and are not meant to limit the scope of the present invention in any way.

### Example 1

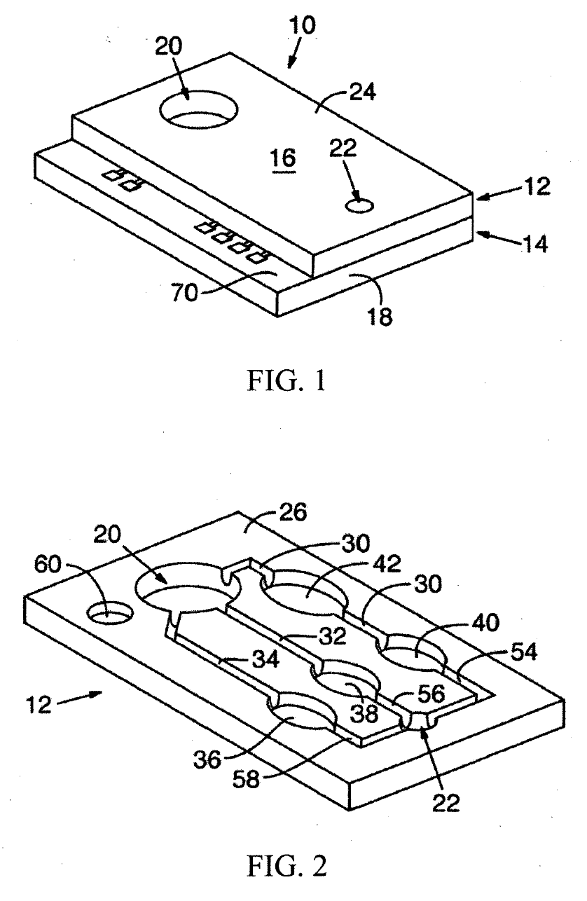

In one embodiment, a method for producing a microfluidic device 10 includes providing a first layer 12 and a second layer 14 as illustrated in FIG. 1. The first layer 12 and the second layer 14 may be formed from the same material. In one embodiment, the material used for the first layer 12 and the second layer 14 are optically transparent, such that light may be transmitted through the microfluidic device 10.

Referring in conjunction to FIGS. 1 and 2, one of the layers such as, for example, the second layer 14 (i.e., the lower layer) has a distribution chamber 20, channels 30, 32, and 34, and analytical chambers 36, 38, 40 and 42 formed in a surface 26 of the second layer 14. In another embodiment, an air exit port 22 may also be formed in order to help the sample be drawn into the microfluidic device 10. It will be apparent to those of ordinary skill in the art that the configuration of the distribution chamber, channels and analytical chambers of the various embodiments discussed herein is merely exemplary and that any suitable configuration may be used. Depending on the type of material used to form the first layer 12 or the second layer 14, the process used to form the distribution chamber, channels and analytical chambers may vary. For instance, if the material is a silica material, the distribution chamber, channels and analytical chambers may be etched, and if the material is a plastic material, the distribution chamber, channels and analytical chambers may be molded into the surface.

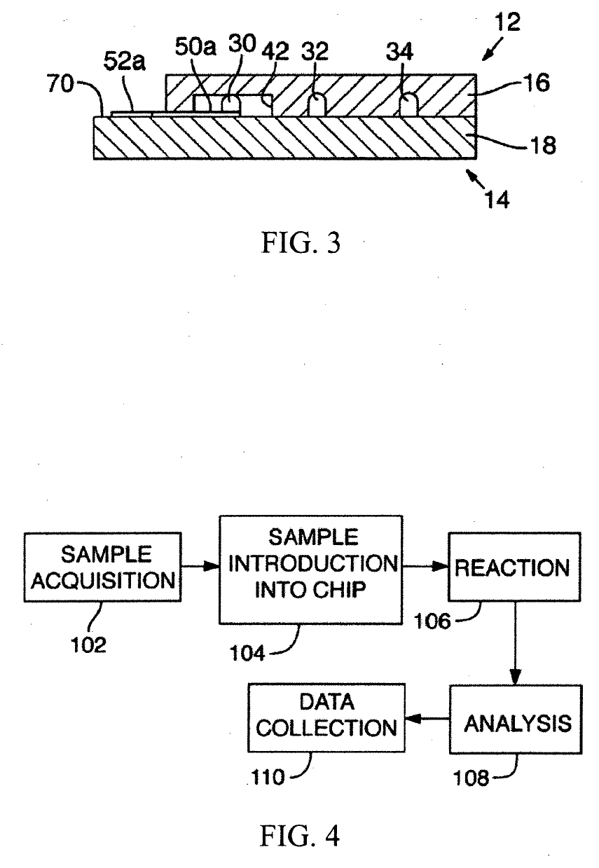

In another embodiment, after the formation of the distribution chamber 20, channels 30, 32 and 32, and analytical chambers 36, 38 and 40 in the second layer 14, various types of sensors 50 (FIG. 3), metallic layers or circuit traces 52 (FIG. 3) may be formed or placed therein for performing various types of analytical tests.

The depressions in the surface 26 of the second layer 14 are coated with a coating. In one embodiment, a method of coating includes mixing polyvinyl alcohol with water in a container (such as a beaker) to produce the coating, and applying the coating on the surface 26 of the second layer 14 including the depressions (i.e., the distribution chamber 20, channels 30, 32 and 32, and analytical chambers 36, 38 and 40). The coating may be applied with a micropipette, a pipette, jetted with an ink-jet pen, blanket deposited, dip coating, layered, or combinations of any thereof.

In other embodiments, the coating may include materials other than or in addition to the polyvinyl alcohol. For instance, when multiple depressions are employed, different materials may be used for different depressions such as using polyvinyl alcohol for a first depression and using vinyl acetate for a second depression. Further, different depressions may have different functions and, thus, different materials used for coating. For instance, a first depression may be used to pre-condition a sample, wherein the sample, once pre-conditioned, travels to a second depression for analysis with a detectable marker.

The deposited coating is allowed to set or dry, thus, forming a coating. In one embodiment, the coating is heated or baked at a low temperature in order to drive the water out of (i.e., dehydrate) the coating and cause the polyvinyl alcohol to gel, thus forming a homogenous clear film of the coating a few microns thick. The concentration of the polyvinyl alcohol with the water in the coating may be varied depending on a desired thickness of the coating. In one embodiment, the coating is formulated to produce a thickness of about 10-50 μm, about 95 μm, or any other desired thickness.

A detectable marker or a reagent such as, for example, a pH indicator is liberally applied to the coating, wherein the coating swells and absorbs the detectable marker. The detectable marker is absorbed by the coating such that the detectable marker is contained within or held by the coating. The detectable marker may be applied to the coating by jetting with an ink jet pen, micropipetting, a pipette, blanket deposition, dip coating, layering, or combinations of any thereof. In one embodiment, the detectable marker is water soluble and used for chemical analysis. In another embodiment, the detectable marker or the reagent may be admixed with the coating such that the coating, and the detectable marker or the reagent are applied to the microfluidic chamber 10 substantially simultaneously. In this manner, the step of separately adding the detectable marker or the reagent to the coating may be omitted, thus, making the method of fabricating the microfluidic chip 10 faster.

In yet an additional embodiment, once the coating and the detectable marker are deposited on the surface in the microfluidic device 10, the first layer 12 and the second layer 14 of the microfluidic device 10 may be oriented in a face-to-face manner in order to encapsulate the surface 26 of the first layer 14, thus, forming the microfluidic device 10 as illustrated in FIG. 3. The first layer 12 and the second layer 14 may be bonded together in any appropriate manner such as, for example, through the use of non-water soluble adhesives, encapsulation, thermal compression, or the use of a polyamide and/or thermoset film.

In various embodiments, the size and scale of the microfluidic device 10 will vary depending on the intended use of the microfluidic device 10. In the exemplary embodiment, the first layer 12 and the second layer 14 are optically transparent, but in other embodiments and depending on the intended use of the microfluidic device 10, only one of the first layer 12 and the second layer 14 may be optically transparent. Further, a thin reflective film may be deposited onto a surface of at least one of the first layer 12 and the second layer 14 of the microfluidic device 10 in order to assist in scattering light.

### Example 2

In another embodiment, the microfluidic device 10 produced using the exemplary method of Example 1 is used to perform an analysis of a sample, such as, for example, assaying the pH of a sample using the microfluidic device 10. FIG. 4 illustrates one embodiment of a method of performing an analysis on a sample. In the method, a sample, such as, for example, a water sample is acquired at box 102 and the water sample is introduced into the distribution chamber 20 of the microfluidic device 10. Capillary action draws the water sample through the channels 30, 32 and 34 and into the various analytic chambers 36, 38 and 40, such that the analysis may be performed.

The coating holds the detectable marker (e.g., the pH indicator) in place as the water sample travels through the various channels 30, 32 and 34 and analytic chambers 36, 38 and 40 of the microfluidic device 10. In this manner, the detectable marker is not moved or transported into other areas of the microfluidic device 10, but rather the detectable marker is anchored in the coating such that the detectable marker does not react with the water sample at box 106 in areas of the microfluidic device 10 other than in desired areas. An analysis of the reaction occurs at box 108 and collection of the data from the analysis occurs at box 110.

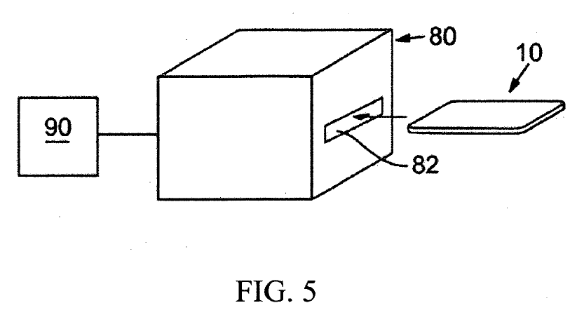

In the exemplary embodiment, the detectable marker is a pH indicator that produces a color change at various pH ranges. Thus, a pH analysis may be performed. The microfluidic device 10 may be used in conjunction with an analytical instrument 80, as illustrated in FIG. 5. In the exemplary embodiment, the analytical instrument 80 includes optical components suited for detecting calorimetric changes in the water sample placed in the microfluidic device 10. The analytical instrument 80 further includes components for reporting the results of the analysis in the form of data that may be saved in internal memory of the analytical instrument 80, and/or the data may be output to a computer 90. In the exemplary embodiment, the analytical instrument 80 may comprise a spectrophotometer or any other device capable of measuring color, absorbance or reflectance.

Although the present invention has been shown and described with respect to various exemplary embodiments, various additions, deletions, and modifications that are obvious to a person of ordinary skill in the art to which the invention pertains, even if not shown or specifically described herein, are deemed to lie within the scope of the invention as encompassed by the following claims. Further, features or elements of different embodiments may be employed in combination.

What is claimed is:

1. A microfluidic device, comprising:
a substrate having a surface;
at least one depression formed in the surface of the substrate;
a coating covering the surface of the substrate; and
at least one detectable marker anchored in the coating.

2. The microfluidic device of claim 1, further comprising at least one sensor located in the depression.

3. The microfluidic device of claim 1, further comprising a sample.

4. The microfluidic device of claim 1, wherein the coating comprises a material selected from the group consisting of: polyvinyl alcohol; albumin; gelatin; casein; starch; gum arabic; sodium or potassium alginate; hydroxyethylcellulose; carboxymethylcellulose; α-, β-, or γ-cyclodextrin; saponified products of copolymers of vinyl acetate; (meth)acrylic acid; maleic acid; crotonic acid; vinylsulfonic acid; styrene sulfonic acid; (meth)acrylamide; homopolymers or copolymers of other monomers with ethylene oxide; polyurethanes; polyacrylamides; water-soluble nylon-type polymers; polyvinyl pyrrolidone; polyesters; polyvinyl lactams; acrylamide polymers; substituted polyvinyl alcohol; polyvinyl acetals; polymers of alkyl and sulfoalkyl acrylates and methacrylates; hydrolyzed polyvinyl acetates; polyamides; polyvinyl pyridines; polyacrylic acid; copolymers with maleic anhydride; polyalkylene oxides; methacrylamide copolymers; maleic acid copolymers; and combinations of any thereof.

5. The microfluidic device of claim 4, wherein the coating further comprises a pigment.

6. The microfluidic device of claim 4, wherein the coating has been crosslinked.

7. The microfluidic device of claim 1, wherein the coating comprises a layer of polyvinyl alcohol is about 10 to 50 microns thick.

8. The microfluidic device of claim 1, wherein the at least one depression comprises an analytic chamber and a channel formed in the surface of the substrate, wherein the analytic chamber is operatively connected to the channel.

9. The microfluidic device of claim 1, wherein the at least one detectable marker is water soluble.

10. The microfluidic device of claim 1, wherein the microfluidic device is operatively connected to an analytic device.

11. A method for producing a microfluidic device, the method comprising:
forming at least one depression in a surface of a substrate;
placing a coating on at least a portion of the surface of the substrate; and
anchoring a detectable marker in the coating.

12. The method according to claim 11, wherein forming the at least one depression comprises forming an analytical chamber, a channel or a combination thereof in the surface of the substrate.

13. The method according to claim 11, further comprising placing a sensor in the at least one depression formed in the surface of the substrate.

14. The method according to claim 11, further comprising placing a sample in the at least one depression.

15. The method according to claim 11, wherein the coating comprises a material selected from the group consisting of: polyvinyl alcohol; albumin; gelatin; casein; starch; gum arabic; sodium or potassium alginate; hydroxyethylcellulose; carboxymethylcellulose; α-, β-, or γ-cyclodextrin; saponified products of copolymers of vinyl acetate; (meth)acrylic acid; maleic acid; crotonic acid; vinylsulfonic acid; styrene sulfonic acid; (meth)acrylamide; homopolymers or copolymers of other monomers with ethylene oxide; polyurethanes; polyacrylamides; water-soluble nylon-type polymers; polyvinyl pyrrolidone; polyesters; polyvinyl lactams; acrylamide polymers; substituted polyvinyl alcohol; polyvinyl acetals; polymers of alkyl and sulfoalkyl acrylates and methacrylates; hydrolyzed polyvinyl acetates; polyamides; polyvinyl pyridines; polyacrylic acid; copolymers with maleic anhydride; polyalkylene oxides; methacrylamide copolymers; maleic acid copolymers; and combinations of any thereof.

16. The method according to claim 15, further comprising subjecting the coating to heat.

17. The method according to claim 11, wherein placing the coating on the at least a portion of the surface of the substrate comprises at least one of micropipetting the coating on the at least a portion of the surface, dispensing the mixture on the at least a portion of the surface with an ink jet pen, spin coating, blanket deposition, and patterning the coating.

18. The method according to claim 11, wherein the detectable marker is water soluble.

19. The method according to claim 11, further comprising encapsulating the coating and the detectable marker with a second substrate.

20. The method according to claim 11, wherein anchoring the detectable marker in the coating comprises mixing the detectable marker in the coating before the coating is placed on the at least a portion of the surface of the substrate.

21. A method for analyzing a sample, the method comprising:
forming a depression in a surface of a substrate;
placing a coating on at least a portion of the surface of the substrate;
anchoring a detectable marker in the coating;
placing a sample in the depression; and
analyzing in the sample with the detectable marker.

22. The method according to claim 21, further comprising operatively connecting the microfluidic device to an analytical device.

23. The method according to claim 21, further comprising encapsulating the coating and the detectable marker with a second substrate.

24. The method according to claim 21, wherein analyzing the sample comprises measuring reflectance, absorbance or scattering of light.

25. The method according to claim 21, wherein anchoring the detectable marker in the coating comprises mixing the detectable marker in the coating before the coating is placed on the at least a portion of the surface of the substrate.
